(19) United States
(12) Patent Application Publication
(10) Pub. No.: US 2006/0079947 A1
(43) Pub. Date: Apr. 13, 2006

(54) Methods and apparatus for modulation of the immune response using light-based fractional treatment

(76) Inventors: Nikolai I. Tankovich, San Diego, CA (US); Basil M. Hantash, East Palo Alto, CA (US); John F. Black, San Mateo, CA (US)

Correspondence Address: FENWICK & WEST LLP, SILICON VALLEY CENTER, 801 CALIFORNIA STREET, MOUNTAIN VIEW, CA, 94041, US

(21) Appl. No.: 11/238,506

(22) Filed: Sep. 28, 2005

Related U.S. Application Data
(60) Provisional application No. 60/613,303, filed on Sep. 28, 2004.

(51) Int. Cl. A61N 5/06

(52) U.S. Cl. 607/89

(57) ABSTRACT

The invention describes a method and apparatus for the modulation of an immune response for the removal of a foreign body in skin or a visceral organ. A fractional phototherapy device is used to produce an acute stimulus for the immune response. The immune response may be enhanced by applying an exogenous substance. The immune modulation may be targeted or directed toward the treatment of a particular foreign body through the creation and amplification of particular biological signatures. This invention is particularly appropriate for treatment of skin cancer, autoimmune diseases, inflammatory diseases, and fungi.

### CROSS-REFERENCE TO RELATED APPLICATION

This application claims priority under 35 U.S.C. §119(e) to U.S. Provisional Patent Application Ser. No. 60/613,303, “Photo-Fractional Immune Modulation Device and Method,” filed Sep. 28, 2004. The subject matter of the foregoing is incorporated herein by reference in its entirety.

### FIELD OF THE INVENTION

This invention relates generally to medical laser and light emitting systems. More specifically, it relates to treating human skin or visceral organs using a fractional phototherapy device to modulate the human immune system through a biological signature and directed amplification with or without introduction of one or more selected exogenous agents.

### BACKGROUND OF THE INVENTION

Medical laser treatments are commonly performed for vascular lesion removal, pigmented lesion removal, skin rejuvenation, tattoo removal, wrinkle reduction, etc. These common laser treatments are not directed towards the stimulation and direction of an immune response to attack a foreign body. For example, laser tattoo removal is accomplished by killing cells or groups of cells that contain or encapsulate tattoo ink particles. The tattoo particles are then flushed out of the skin by the lymphatic system in the absence of a concerted immune response directed to remove the tattoo ink particles.

Similarly, for laser treatment of dyschromia, melanocytes, keratinocytes, and melanophages are coagulated and the melanin is carried away as the coagulated cells are exfoliated or washed away by the lymphatic system. The removal of these cells occurs through a process that does not involve the immune system targeting the melanin or the melanin containing cells.

For laser skin rejuvenation, a laser can be used to stimulate a wound healing response by killing cells and coagulating tissue to stimulate collagen synthesis. The collagen remodeling is directed by fibroblast stimulation and subsequent collagen production does not involve the immune system.

There has been a reluctance to use lasers for treatment of cancer because there is a perceived risk of incomplete removal of the cancer cells, which may later metastasize and cause a fatal outcome. Standard treatments for cancers of the skin and visceral organs include cryotherapy and surgical removal. Both of these procedures are invasive, can leave scars, can discolor skin, and can leave behind residual cancer cells. Ineffective cancer treatments require follow-up procedures, such as chemotherapy and radiation therapy, that cause serious side effects such as liver, gastrointestinal, and bone marrow toxicity, with the latter leading to immune system suppression. This is in addition to the laundry list of less serious side effects such as hair loss and nausea. Neither surgery nor cryotherapy offers predictable cure rates, and metastasis is not uncommon despite follow-up with radiation or chemotherapy. To avoid ineffective treatment, physicians generally perform wider excisions and more aggressive adjunctive therapy in order to prevent the potentially life threatening outcome of metastasis. An effective cancer treatment that is less invasive is desired.

Non-fractional ablative lasers are not typically used for removal of foreign bodies such as cancer cells or tattoo ink particles that lie within the dermis because of the risk of excessive scarring. Nonablative lasers, such as Q-switched Nd:YAG lasers, can be used to remove some foreign bodies by breaking up the foreign body and reducing its size enough to allow the lymphatic system to carry it away in piecemeal. Nonablative lasers have not been used to remove a living foreign body such as a tumor because these lasers are not typically turned up to high enough fluence to cause necrosis of the cells. If these lasers are turned up high enough, then they cause the undesirable levels of scarring similar to the scarring caused by ablative lasers. The removal of foreign bodies through light-based stimulation of the immune system has not been described. It is desirable to use the immune system to assist in the removal of foreign bodies with higher efficacy, with less scarring, and/or with less invasiveness.

Fractional light-based treatment modalities have been described for treatment of wrinkles and other indications. Due to sparing of tissue around each treatment zone, fractional light-based treatments allow higher local fluence levels to be used without scarring than large area nonablative lasers. In addition to the general reluctance cited above for the use of light-based devices for cancer treatment, there has been a particular resistance to use fractional treatment due to the perceived partial tissue coverage of this type of treatment. Apparatus and methods have not been designed to use the temporal and/or spatial signatures of fractional light-based treatment to enhance or suppress the immune system response.

Light-based treatments have been combined with exogenous agents to obtain a particular immune response. Apparatus and methods have not been designed to use the temporal and/or spatial signatures of light-based treatments in combination with exogenous agents to enhance or suppress the immune system response that is caused by the exogenous agent.

No one has directed the resensitization of a suppressed or blocked immune system using fractional light based treatments or using light based treatments in combination with pharmaceutical agents.

What is needed is a method and apparatus for stimulating the immune system to assist with the removal of foreign bodies and/or the treatment of cancer and inflammatory diseases in skin and visceral organs. Inflammatory diseases include but are not limited to infection and autoimmune diseases.

Several medical conditions result in lack of sensitivity by the immune system to a particular foreign presence in the body. The most important example is the presence of cancer, which can be fatal. Cancer cells frequently create an environment that promotes the growth of cancer cells while suppressing the activation of cytotoxic T-lymphocyte cells, which are a key part of the human immune response that would otherwise carry out the destruction of the cancer. The suppression of T-lymphocyte cells renders these cells unavailable in defeating the growth of new cancer cells leaving them to proliferate unchecked and eventually metastasize. Cancer remains one of the leading causes of death in the United States each year.

Cancer is typically treated with a multi-therapeutic approach incorporating the use of toxic systemic chemotherapy, radiation, surgical excision, and electrocautery. Frequently, these therapeutic regimens only transiently stabilize the rate of cancer growth while the cancers cells continue to mutate with each division, eventually rendering them resistant to the treatment modality. These therapeutic options also suffer from systemic side effects which inherently place the patient at risk of significant morbidity and possible mortality. For example, some of the side effects relate to immune suppression and involve a variety of systemic infections during the immunocompromised state. Therefore, a new approach that stimulates the immune system to respond to a foreign body while avoiding systemic toxicity is needed.

Other light based treatment modalities are used to suppress the immune system. For example, ultraviolet light has been used to suppress the immune system in ivitiligo, atopic dermatitis, psoriasis, and mycosis fungoides. Ultraviolet light has also been shown to suppress Langerhans cell function. There has been no evidence or suggestion that uniform illumination with ultraviolet light is sufficient to stimulate the immune system to fight cancer or dispose of foreign bodies. In fact, long-term ultraviolet light exposure has been linked to development of a variety of skin cancers as a result of creation of mutagens and chronic immune suppression. A device is needed that allows selective suppression and stimulation of the immune system with a reduced risk of forming mutagens.

There is indirect and direct evidence that laser-tissue interaction leads to partial immune stimulation with a variety of cytokines upregulated immediately after laser treatment of skin. However, light-based treatments have not been used to stimulate the immune system because they are broad area treatments. A limitation of broad area light-based treatments is that heating destroys some of the signal pathways necessary to orchestrate an appropriate immune response. Other light based treatments use low energies to avoid the destruction of the signal pathways, but these treatments do not create substantial stimulation of the immune system.

Anderson et al. (U.S. patent application Ser. No. 10/033,302) and Manstein et al. (PCT patent application PCT/US04/09452) disclose the use of light based therapies for the treatment of the skin and for the stimulation of wound healing response. However, these devices have not been targeted to stimulate the immune system selectively. The first phase of the wound healing response involves increasing vascularization to the injured site. However, this alone is inadequate to remove foreign bodies such as cancers. In fact, increasing angiogenesis may promote cancer growth. Promotion of wound healing alone is insufficient and a method to promote the immune system without turning on cancer promoting signals is needed.

Thus there is a need for methods and apparatuses to stimulate the immune system to attack foreign bodies present in the skin and the visceral organs of the patients. The present invention addresses the deficiency in the prior art and provides non-invasive and non-toxic methods and apparatuses for the modulation of immune responses. The inventive methods and apparatuses thus overcome the problems of systemic chemotherapy which is fraught with systemic side effects.

### SUMMARY OF THE INVENTION

The invention uses fractional phototherapy devices that provide acute stimulus thereby modulating the immune system. The invention provides methods whereby an immune response can be modulated by an acute stimulus provided by a fractional phototherapy device in order to achieve a desired treatment outcome. A fractional phototherapy device can be used in order to spare tissue between the injury zones that allows aspects of the immune response to be amplified and/or suppressed through interaction with vascular and cellular structures of the target tissue.

The fractional phototherapy device can emit treatment wavelengths in the range of about 180 nm to about 28000 nm, such as, for example, 190-400 nm, 400-28000 nm, 1300-1900 nm, or 2000-2400 nm. The optical source of the treatment energy for the fractional phototherapy device may be a laser such as for example, a Nd:YAG, CO₂, holmium, Er:YAG, and/or Er:glass laser. The optical source can alternatively be a flash-lamp, a dye laser, a fiber laser, and/or a diode laser. The injury zones created by the fractional phototherapy device can have aspect ratios of depth:width of in the range of about 3:1 to about 10:1 and/or surface area:volume ratios in the range of about 20,000-100,000 m⁻¹.

Embodiments of the invention are directed to the treatment of inflammatory disease in visceral organs, inflammatory skin disease, noninflammatory skin disease, acne, psoriasis, alopecia areata, and/or vitiligo. In some cases, the stimulation of the immune system can be systemic.

In one embodiment of the invention, the immune system can be modulated and/or directed to attack one or more foreign bodies in a region of the skin or in a visceral organ, wherein the immune system removes a foreign body after immune cells are recruited and mobilized. In another embodiment, the foreign body is only partially removed or is changed in character. Examples of foreign bodies that can be attacked include skin cancer, tattoo ink particles, microbial infection, fungal infection, autoimmune disease cells, and/or human papilloma virus.

In another aspect of the invention, the treatment with a fractional phototherapy device can be performed in addition to surgical removal of at least part of a foreign body. At least part of the foreign body may be located within the dermis.

In another embodiment of the invention, an exogenous agent can optionally be used in conjunction with the stimulation by the fractional phototherapy device to direct or selectively modify the immune response. The exogenous agent can be introduced to the surface of the target tissue or may be introduced by the circulatory system. Examples of exogenous agents include stem cells, homing molecules, targeted stem cells, autologous immunotoxic cells, immunomodulators, vaccines, cytokines, growth factors, paracrine molecules, and/or chemotactic factors. Examples of autologous immunotoxic cells that can be used are natural killer cells and cytotoxic cells. Targeted stem cells are stem cells which are capable of being directed to a particular location in the body. Targeted stem cells can be created by genetically altering stem cells such that they express homing markers that bind to topographic specific markers thus enabling the stem cell to be directed to a particular location in the body.

In another embodiment of the invention, surface cooling is used to create a thermal inversion within the target tissue. Multiple passes may be made at an interval of 0.5 to 10 minutes. Multiple treatments can be used spaced at 1-2 week intervals.

In another aspect of the invention, only the periphery of a region containing a foreign body or presenting an undesirable condition can be treated.

Fractional treatment can create a homing signal for directing the immune system to a particular location. In this way, the dominant component of the immune response can be localized within 1 cm of the area treated by the fractional phototherapy device. In one aspect of the invention, method is provided for modulating the immune response of a subject. The method can comprise contacting a fractional phototherapy device with a target tissue wherein the immune response is modulated, and wherein the target tissue is skin or a visceral organ. The fractional phototherapy device can emit energy with a wavelength of about 400 nm to about 28,000 nm, of about 190 nm to about 400 nm, of about 1300 nm to about 1900 nm, or of about 2000 nm to about 2400 nm. The fractional phototherapy device can be a erbium-doped fiber laser.

Other aspects of the invention include apparatuses corresponding to the methods described above. These and other aspects of the present invention will become evident upon reference to the following detailed description.

### BRIEF DESCRIPTION OF THE DRAWINGS

The invention has other advantages and features which will be more readily apparent from the following detailed description of the invention and the appended claims, when taken in conjunction with the accompanying drawings, in which:

FIG. 1 depicts the modulation of the immune response using a fractional phototherapy device for the removal of a foreign body within the skin or a visceral organ. In FIG. 1A, the optional exogenous agent (121) is introduced near the foreign body (151). In FIG. 1B, a fractional phototherapy device (100) is applied creating the injury zones (110) and the untreated tissue (112), and to modulate the immune response. In FIG. 1C, the foreign body is removed by the treatment.

FIG. 2 is a graph that shows the variation of the dimensions of the injury zones for a particular fractional phototherapy device that can be used in some aspects of this invention.

FIG. 3 shows the acute cellular-derived inflammatory response that can be created in response to application of a fractional phototherapy device.

### DETAILED DESCRIPTION

### DEFINITIONS

Immune cells are cells that help protect the body against anything perceived as foreign by the host. Examples of immune cells include T lymphocytes, B lymphocytes, macrophages, and natural killer cells (NK cells).

An injury zone is a contiguous region of tissue that is coagulated by a fractional phototherapy device.

An inflammation zone is a region around an injury zone in which an inflammatory response is triggered by treatment with a fractional phototherapy device. The size or character of the inflammation zone may be affected by the treatment energy delivered by the fractional phototherapy device or by the presence or absence of an exogenous agent. The inflammation zone does not comprise the injury zone.

“Untreated tissue” refers to tissue not part of the injury zone.

Exogenous agents are agents that come from outside the body. Exogenous agents include agents that are derived from the body and later reintroduced into the body, such as autologous stem cells, for example.

As used herein, the terms “treat” or “treatment” are used interchangeably and are meant to indicate a postponement of development of diseases and/or a reduction in the severity of such symptoms that will or are expected to develop. The terms further include ameliorating existing symptoms, preventing additional symptoms, and ameliorating or preventing the underlying metabolic causes of symptoms.

The term “optional” or “optionally” means that the subsequently described event or circumstance may or may not occur, and that the description includes instances where the event or circumstance occurs and instances where it does not. For example, the phrase “optionally another drug” means that the patient may or may not be given a drug in conjunction with the fractional phototherapy described herein.

The term “modulating” refers the inhibition or promotion of the activity of the immune response or concentration of any enzyme or regulatory molecule involved in the immune response in a cell or animal. Modulator can be a fractional phototherapy device, a polypeptide, nucleic acid, macromolecule, complex, molecule, small molecule, compound, or the like (naturally occurring or non-naturally occurring) that is capable of causing modulation.

### DEFINITIONS

In some diseases of the skin or visceral organs, the unassisted immune response has failed to respond adequately. Therefore, treatment can be affected by appropriately stimulating the target region in order to reprogram the immune system to once again recognize foreign bodies that cause the skin disease. Examples of such foreign bodies include malignant cells, viruses, fungi, microbes, or autoimmune cells and antibodies, amongst others. The treatment of each of these foreign bodies can be performed through combining a fractional phototherapy device with topical immunomodulators and/or vaccines. In other situations, the immune system can be hypersensitive and the immune response can be beneficially suppressed or modified. For example, in inflammatory skin diseases, the inflammatory response typically exceeds desirable levels. The excess inflammatory response can be counterbalanced with the appropriate immunostimulation, for example by blocking undesirable signaling pathways.

The present invention discloses methods and apparatuses for modulating the immune system response to trigger the immune system to attack one or more undesirable foreign bodies within the tissue. In one aspect of the invention, the immune response can be modulated by applying energy. The energy can be light, heat, or mechanical energy, such as, for example, energy provided by UV, visible, infra red or near infrared sources, contact heat, or RF heat, or mechanical energy provided by tapping, messaging, mechanical stimulation, and the like. A preferred embodiment of the invention comprises the use of a fractional phototherapy device to stimulate or suppress the immune system with the optional addition of an exogenous agent that enhances and/or directs a response of the immune system.

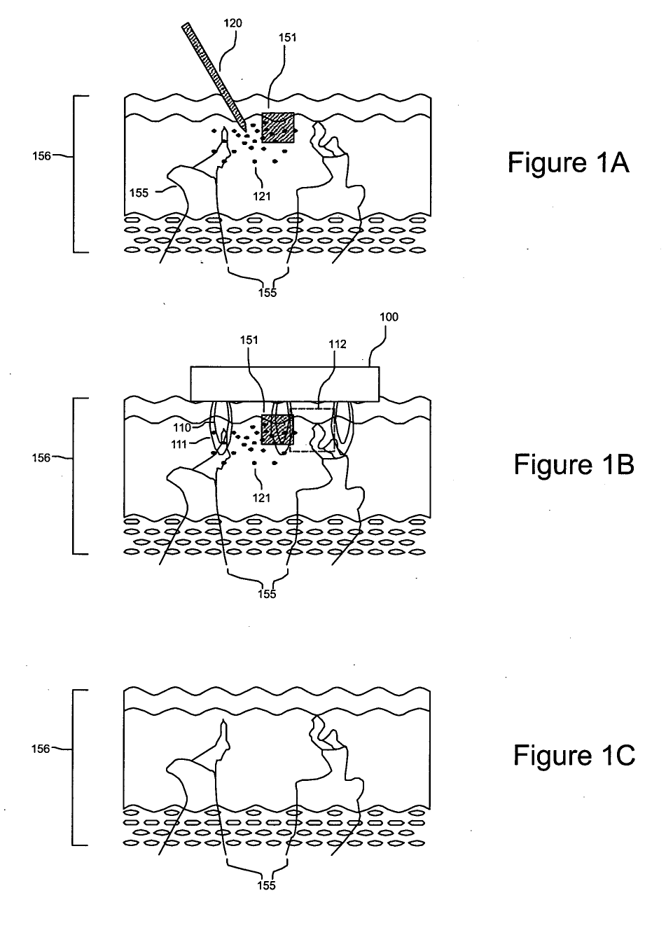

One embodiment of the invention is illustrated in FIG. 1. In FIG. 1A, a needle 120 is inserted into the tissue 156 to inject an exogenous agent 121. Depending on the location of the foreign body 151 that is to be removed and the types of immune cells that will be stimulated, the optional exogenous agent 121 may be injected into a selected layer within the tissue 156. After the exogenous agent 121 has been injected into the tissue 156, the fractional phototherapy device 100 can be used to illuminate the skin to create discrete injury zones 110 as shown in FIG. 1B. Injury zones may reach the surface or may be located entirely below the surface, for example, within the dermis of the skin. Without being bound to theory, the cells within the injury zones 110 are coagulated by the light treatment and some of these cells release cytokines in response to the optical treatment. The released cytokines migrate to the blood vessels 155 and stimulate an immune response that attacks and removes a foreign body 151 as shown in FIG. 1C. Between at least some of the injury zones 110 are inflammation zones 111 wherein the immune system is stimulated by the treatment with the fractional phototherapy device 100 but the tissue 156 is not coagulated. There will be untreated tissue regions 112 between the injury zones 110 wherein the tissue is not coagulated. The untreated tissue regions 112 can include inflammation zones 111.

In one aspect of the invention, the fractional phototherapy device 100 is a FRAXEL™ SR laser system (Reliant Technologies, Inc. Palo Alto, Calif.). The FRAXEL™ SR laser system incorporates an erbium doped fiber laser that operates at a wavelength of 1550 nm and uses a microscopic beam that is nearly collimated as it enters the skin. An example of a fractional phototherapy device 100 is described in co-pending U.S. applications Ser. No. 10/367,582, entitled “Method and apparatus for treating skin using patterns of optical energy” and Ser. No. 10/888,356, entitled “Method and Apparatus for fractional photo therapy of skin” which are incorporated herein by reference.

The use of optical energy is preferred over contact heating or RF energy because optical energy can be better controlled to deliver smaller spot sizes than other energy types. This is beneficial for creation of many patterns of stimulation for the immune response.

In one aspect of the invention, the fractional phototherapy device has a wavelength in the range of about 180 nm to about 30,000 nm, preferably about 400-28,000 nm. The wavelengths in this range can be produced using a flash-lamp, a free electron laser, and/or an optical parametric oscillator pumped by a laser. Other types of laser sources can be used to create light at selected wavelengths within the range of 400-28,000 nm. For example CO₂, erbium-doped fiber, Er:YAG, Er:glass, holmium, dye, thulium-doped fiber, diode, Nd:YAG, and Nd:YAP lasers can be used. Tunable lasers can also be used to allow additional flexibility for treatment. Frequency doubling can also be used, for example, with a Nd:YAG laser.

The fractional phototherapy device can produce injury zones 110 with small diameters or large diameters have an aspect ratio of depth to width of greater than 2:1 or in the range of about 3:1 to about 10:1 are desired. In other embodiments, certain immune responses can be created by using injury zones with a large ratio of surface area to volume such as in the range of about 20,000-100,000 m⁻¹. Larger surface area to volume ratios can advantageously allow a larger percentage of immunomodulators to diffuse from the injury zone to the adjacent viable tissue. To create injury zones 110 with large aspect ratios, large surface area to volume ratios, and/or small diameters, wavelengths that have low scattering and low absorption within the skin are preferred. Such wavelengths occur in the wavelength range from about 1300-1900 nm and in the range of about 2000-2400 nm. Ultraviolet wavelengths such as 190-400 nm are particularly useful for immune suppression in the upper layers of the tissue. The choice of optical source (e.g. laser) and optical wavelength for the fractional phototherapy device can be made by determining the desired depth, width, shape, and location of the injury zones and then using Monte Carlo simulations to model the absorption, scattering, and thermal diffusion within the tissue 156.

Returning to FIG. 1, FIG. 1A shows an exogenous agent 121 in contact with tissue 156. The exogenous agent 121 can be delivered by one or more known methods, such as injected directly to the site using a needle 120, by transdermal delivery, or via systemic delivery using oral, intravenous, or intramuscular delivery. The exogenous agent 121 can also be delivered via topical routes, for example, by application to the surface of tissue 156. As one of skill in the art will recognize, combination methods can be used to deliver one or more exogenous agents 121 to the target region.

The exogenous agent 121 can be, for example, a pharmaceutical agent, a nutraceutical, a drug, a vitamin, an immunomodulator, stem cells, autologous immunotoxic cells, an exogenous immune or other type of cell, or an agent that contains one or more active ingredients that promote immune modulation, or combinations thereof. In some situations, the exogenous agents may be naturally present at levels that are not sufficient to cause therapeutic benefits at the desired level. For example, cellular-derived growth factors may be naturally occurring within the skin, but the concentration of cellular-derived growth factor may provide insufficient stimulation of the immune response to cause the desired treatment.

An advantage of the use of fractional phototherapy device 100 with an exogenous agent 120 delivered by the circulatory system in comparison to the use of the exogenous agent 120 alone is that the treatment with the fractional phototherapy device 100 can direct the immune response to a selected location. For an exogenous agent 120 that has been delivered into the circulatory system, an immune response will be induced in the circulatory system. In the absence of a homing signal, the immune response becomes diluted by traveling throughout the body in a non-selective fashion. This renders the immune response ineffective. Treatment with the fractional phototherapy device 100 can provide a homing signal that triggers the immune response to address the target region.

The invention provides methods and apparatuses for the treatment of a subject having a foreign body 151 located in or on the tissue 156. The tissue 156 is preferably skin, and the subject is preferably human. The method and apparatus of the invention may also be directed towards a foreign body 151 located in tissue 156 of a visceral organ, such as for example, the lungs, heart, liver, kidney, and bladder.

Foreign body 151 may comprise, for example, a cluster of skin cancer cells, tattoo ink particles, pathological inflammatory cells, antigens, and/or infectious agents. Following treatment, the foreign body 151 may be removed fully as depicted in FIG. 1C, may be removed partially, or may be changed only in character by a targeted immune response.

By modulating the immune system through a stimulus created using a fractional phototherapy device 100, a concert of signals will be generated that otherwise would be destroyed or weakened by non-fractional treatment. In one aspect of the invention, a specific biological signature of thermal injury can be created which leads to a very specific response by the immune system. The induced signal can promote immune resensitization locally at the site of injury. The subsequent resensitization can allow the body to reactivate its fight against cancers such as melanoma, basal cell carcinoma, and squamous cell carcinoma, amongst others. Without immune resensitization, the body is unable to recognize the cancer and no immune response is mounted against the harmful cancer cells.

FIG. 1 shows that the exogenous agent 121 is introduced prior to the treatment by the fractional phototherapy device 100. In alternate embodiments, the substance may be introduced during or after the optical treatment with the fractional phototherapy device 100. To achieve synergistic effects on the immune response, a preferred embodiment comprises treatment with the fractional phototherapy device 100 that occurs within 3 or within 12 hours of delivery of the exogenous agent 121 to the target region.

The immune system response can be directed by controllably modulating the balance between cytotoxic CD8 T-lymphocytes and helper CD4 T-lymphocytes. The fractional phototherapy device 100 can be used to stimulate the immune system by increasing the concentration of CD4 cells relative to the concentration of CD8 cells within the target region of tissue 156. Illumination from the fractional phototherapy device 100 can stimulate T-lymphocytes that are CD4+ CD8− in nature. These cells are known as T-helper cells. This immune response promotes the recruitment of eosinophils and other immune cells. Interaction of the T-helper cells with B-lymphocytes stimulates the production of antibodies that attack particular antigens recognized as foreign by the immune system. The antibodies can thus be created to attack a foreign body 151, such as for example cells expressing melanoma, viral, fungal, or bacterial antigens.

In an alternate embodiment, the immune response can stimulate the production of CD4− CD8+ T-lymphocytes which are cytotoxic in nature. These cells are able to effect cellular destruction of antigens perceived as foreign. Cytotoxic stimulation can be used to reintroduce or amplify immune sensitivity in cases where the body does not have adequate sensitivity to a particular foreign body 151 such as for example a cluster of cancer cells or a tattoo.

Each immune response has a particular function and can be regulated. There are times where both CD4+ CD8− and CD4− CD8+ responses can be advantageously increased or decreased. The coordinated regulation of these two types of immune response is not typical since each immune response releases immune mediators that act to inhibit the other arm of the immune system. For example, CD4+ CD8− T-cells are able to generate immune signals such as IL-10 which act to shut down the cytotoxic T-cell response. Similarly, CD4− CD8+ T-cells are able to shut down T-helper cell response through release of a different panel of cytokines. Thus, within each positive stimulatory response, there exists a coordinated suppression of the other arm in most cases. A fractional phototherapy device 100 can be used with or without an optional exogenous agent 121 to create a coordinated regulation of the CD4+ CD8− and CD4− CD8+ types of immune response. By using the spatial signature from a treatment with a fractional phototherapy device to direct the immune response, a particular region of treatment can lead to stimulation of CD4+ CD8− predominant responses in one portion and CD4− CD8+ predominant responses in an adjacent but distinct zone of the same treatment region. Thus, the fractional phototherapy device allows intricate spatial control and direction of the immune response.

In an alternate embodiment, other specialized immune cells may also be selectively activated or suppressed. Examples of these immune cells are natural killer cells, macrophages, monocytes, neutrophils, eosinophils, basophils, mast cells, histiocytes, dendritic cells, and langerhans cells.

Non-fractional phototherapy devices are limited in their ability to predictably achieve particular immune responses. A treatment of a skin cancer that extends from the surface of the tissue 156 to beyond the junction between the epidermis and dermis can be approached using two methods that use a non-fractional phototherapy device: A first method is to kill the cancer cells by heating the cells until they are dead or physically ablated from the skin. This process can be effective but has a high incidence of scarring and can be invasive. A second method is to heat the cells or surrounding cells to sufficient temperature to stimulate an immune response that may remove the cancer cells. With a non-fractional phototherapy device, the intensity of the stimulation that is preferred to provide predictable treatment increases the tissue temperature above 42° C. This thermal condition denatures many large proteins, such as growth factors, cytokines, and paracrine hormones that would otherwise act as immunostimulators. Therefore, when performing treatment with a non-fractional phototherapy device, a large percentage of the proteins within the treatment region can be denatured. The denaturation of a large percentage of particular immunostimulators by a non-fractional phototherapy device blunts much of the expected immune response due to denaturation of the critical immunostimulator molecules necessary to initiate and amplify the immune response cascade.

In another aspect of the invention, methods are provided for using a fractional phototherapy device 100 to stimulate an acute immune response that can be amplified in the inflammation zones 111 and/or in the untreated tissue regions 112 around and/or between the injury zones 110. In some cases of fractional treatment, the inflammation zones 111 from adjacent injury zones 110 can merge together. Fractional treatment can be used to create an acute response as with non-fractional treatment, but the microscopic size of the injury zone 110 allows a higher percentage of active immunostimulators to be available in the inflammation zones 111 and/or in the untreated tissue regions 112 where they can participate in and/or initiate the immune response cascade.

The method of the invention provide for uncoagulated tissue being present in between the injury zones. The presence of uncoagulated tissue in the regions between injury zones 110 preserves native structure of the immune mediator signals that can be released from cells in the target region during the amplification process. Without being bound to theory, the uncoagulated tissue can amplify the immune signal and allow the immune signal to propagate. Amplification and propagation can provide positive feedback for the initial immunostimulation performed by the fractional phototherapy device 100 and thus cause more vigorous host response and improve the likelihood of immune resensitization and subsequent removal a foreign body 151.

Fractional treatment allows higher treatment levels without scarring than would be possible with non-fractional treatment. This can be used to selectively increase the circulation of preexisting immunosurveillance cells.

The percentage of active immunostimulators can be affected significantly by the size of the injury zones 110 and the separation between adjacent injury zones 110. Injury zones 110 with diameters of 50-500 μm or 50-200 μm are preferred in order to allow a significant fraction of the immunostimulators to diffuse out of the injury zones 110. Injury zones 110 will preferably penetrate into the dermis 153 so that the immunostimulators released by the fractional phototherapy device 100 will be able to quickly diffuse to the blood vessels 155 to recruit the desired immune cells to the treatment site.

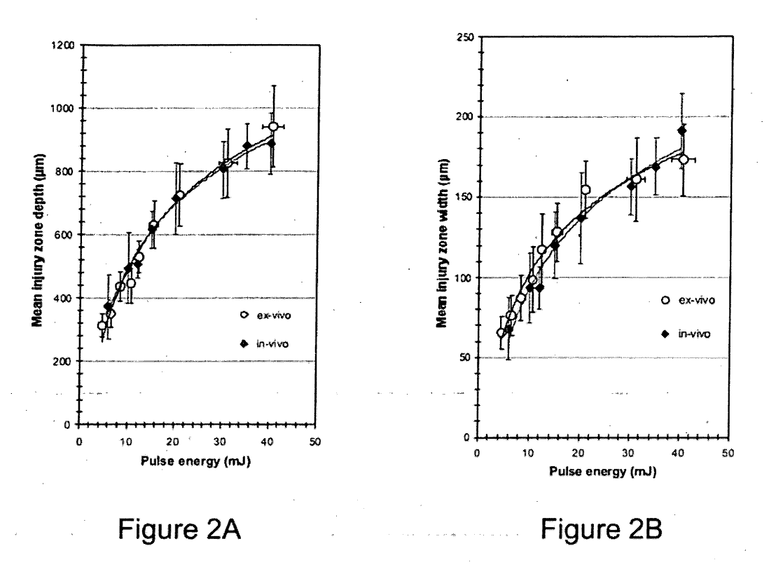

FIGS. 2A and 2B show the average dimensions for injury zones 110 created by the FRAXEL™ SR laser system for in vivo and ex vivo human skin. The average dimensions for injury zones 110 created by the FRAXEL™ SR laser system can vary in width in the range of approximately 50-200 μm and depth in the range of approximately 350-900 μm. Injury zones 110 of other dimensions can be created using other optical configurations.

Using a fractional pattern of delivery allows each biological signature to be controlled temporally and spatially, thus permitting a unique form of dosimetry. Immunostimulation can therefore be tailored to each foreign body 151. Specific immune responses can be generated by appropriately choosing the parameters of the fractional phototherapy device 100. These parameters can include pulse energy, separation between injury zones 110, density of injury zones 110, cooling of the surface of tissue 156, and diameter, depth, shape, and aspect ratio of each injury zone 110. In a preferred embodiment, treatment parameters can be chosen to generate a wide array of biological signatures that mimic the biological signatures of immune responses to different types of pathological conditions. A biological signature is a specific set of immune mediators at specific concentrations. The ability to create a selected biological signature during thermal injury of skin is made possible by the presence of uncoagulated tissue between the injury zones 110. In the absence of tissue sparing at the microscopic level, much of the response would be abolished or blunted. The fractional phototherapy device 100 can create biological signatures that are not part of the wide array created by the human body. This feature provides a novel mechanism to selectively activate unique non-physiological signatures advantageous to effecting removal of foreign bodies.

The immune mediators which constitute the biological signature include cytokines such as TNF-alpha, IFN-gamma, IL-1, IL-2, IL-4, IL-5, IL-8, IL-10, IL-12; growth factors such as TGF-beta1, TGF-beta3, VEGF, PDGF, KGF, FGF, stem cell growth factor; paracrine molecules such as histamine, bradykinin, substance P; and chemotactic factors such as C5a, ECP, and LTB4. Each particular set of treatment parameters will generate a biological signature. The biological signature can be tailored to the foreign body 151 present. This allows the user to resensitize the immune system to a foreign body 151. The treatment with a fractional phototherapy device 100 may be combined with an exogenous agent 121 such as for example efudex or imiquimod to further increase the intensity of the host response.

The biological signature can be enhanced by providing one or more subsequent treatments with the fractional phototherapy device 100, with an exogenous agent 121, or with a combination of the two. The subsequent treatments can create a second set of injury zones and stimulate the release of a second set of cytokines that can enhance an immune response through amplification.

The biological signature can also be enhanced by optionally applying non-fractional variations of thermal profiles within the layers of the tissue 156. For example, a mild uniform heating of 5-10° C. applied at the surface of tissue 156 can be used to provide additional stimulation of selected immune responses. There can also be a temporal thermal signature that is used to heat or cool the tissue as the immune system reacts in order to better facilitate healing. Cycles of heating and cooling may also be used to enhance the immune modulation. In some tissue conditions, CD4+ and CD8+ cells may thrive at different temperatures. In these cases, the balance between CD4+ and CD8+ cells can be controlled by varying a controlled temperature profile following treatment with a fractional phototherapy device 100. The controlled temperature profile can be fractional or non-fractional and can vary with depth into the tissue as a function of time.

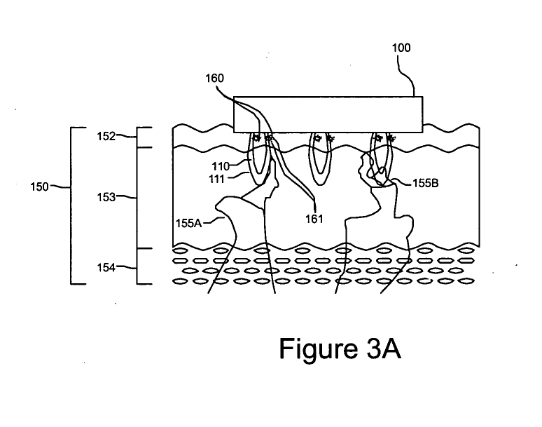

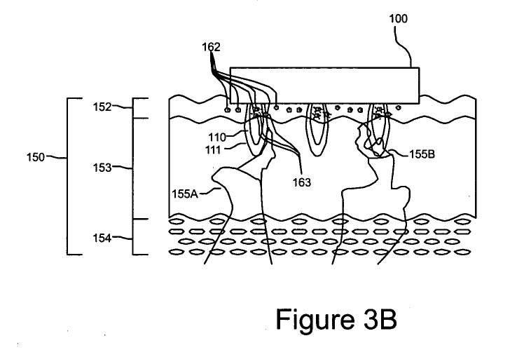

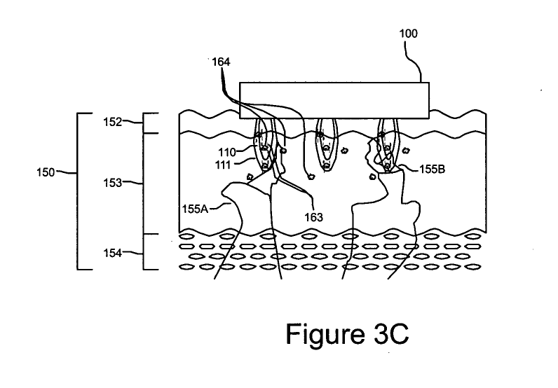

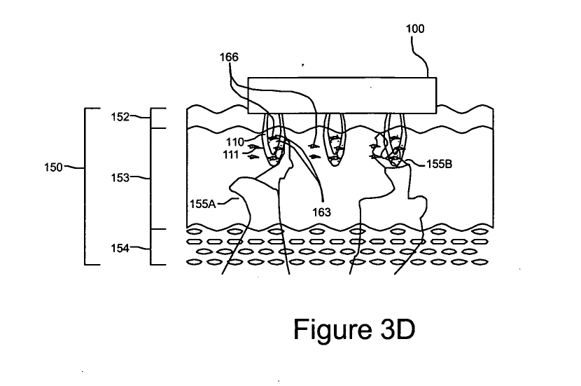

An immune response can be induced within the tissue itself by activating preexisting resident immune cells such as Langerhans cells, dendritic cells, macrophages, histiocytes, and mast cells. For example, FIGS. 3A, 3B, 3C, and 3D show the response of Langerhans cells 160, epithelial cells 162, macrophages 164, and fibroblasts 166, respectively. The responses illustrated in FIGS. 3A-3D are shown separately for clarity. In practice, multiple responses can be stimulated simultaneously by the acute injury zone created by a fractional phototherapy device.

FIG. 3A shows Langerhans cells 160 within the epidermis 152 of skin 150. Some of these Langerhans cells 160 are affected by the fractional phototherapy device 100, while others are not affected. The affected Langerhans cells 160 can release, for example, cytokines 161. FIGS. 3B, 3C, and 3D show the response of epithelial cells 162, macrophages 164, and fibroblasts 164 within the epidermis 152 and/or dermis 153. In each of these cases, cells are coagulated by a treatment with a fractional photothermal device 100 release cellular derived growth factors 163 that can stimulate or suppress an immune response.

The response of the Langerhans cells 160, epithelial cells 162, macrophages 164, and/or fibroblasts 164 can be amplified or suppressed in the presence of an exogenous agent 121.

In one embodiment, the foreign body 151 can be a cluster of cancer cells. A fractional phototherapy device 100 can treat a target region that comprises the cancer cells to produce a stimulus that is co-localized within the target region and thus provides a homing signal for directing the immune response. By thus co-localizing the immune system response to the site of cancer, the host immune response can attack and destroy the skin cancer. The immune response can thus be predominantly located within 1 cm of the treatment to provide a targeted response of the immune system.

In another preferred embodiment, the foreign body 151 is a cluster of skin cancer cells. The fractional device 100 can be used to treat the target area subsequent to a first treatment course of cryotherapy, surgery, and/or laser surgery. The fractional phototherapy device 100 can be used to stimulate an immune response to attack any cancer cells that are not removed by the first treatment course. The use of the fractional phototherapy device 100 after the first treatment course has the benefit of providing additional therapy that reduces the chance of not treating or removing all of the cancer cells of the cluster of skin cancer cells and thus reduces the incidence of residual cancer cells metastasizing. The treatment provided by the fractional phototherapy device 100 can be noninvasive and may supplement a more invasive first treatment course to allow the first treatment course to be made less invasive. Bulk laser treatment can be used in certain cases where a general immune system response is desired. In many cases, fractional treatment is preferred over non-fractional treatment because fractional treatment can provide a better tolerated acute immune stimulus and allows the immune response to be amplified by the tissue outside each injury zone.

Basal cell carcinoma is the most common cancer in both sexes. Only relatively superficial basal cell carcinomas can be treated topically. For example, 5-fluorouracial or imiquimod can be applied topically or injected. A limitation of topical application is that these medications are unable to reach deeper tissues and thus are not considered reliable for removal of basal cell carcinomas that penetrate deeper than the dermal-epidermal junction. A fractional phototherapy device can be used to provide an acute immune stimulus that can be combined with topical therapies to produce an immune response that is effective for removing skin cancers that extend deeper into the tissue 156 than could be addressed by topicals alone. Since only a fraction of the skin is treated, the incidence of side effects is reduced.

The combination treatment can be further enhanced by creating an inverted thermal profile in which the temperature of deeper tissue is increased, while the temperature of shallow tissue is increased by a smaller amount or reduced in temperature by using surface cooling. The inverted thermal profile can provide significant immune system stimulation deeper within the tissue where topical penetration is typically low. The immunostimulation can be varied by adjusting optical pulse fluence, optical pulse duration, optical power, pulse interval, and/or separation between treatment zones. In one aspect of the invention, the temperature profile within the skin can be adjusted through the adjustment of the timing, amount, and location of tissue cooling and heating. The tissue may, for example, be cooled with a cryogenic spray or heated with a resistive heater that is placed in thermal contact with the tissue 156. Thus, the immune stimulation provided by treatment with the fractional photothermal device 100 can be adjusted to achieve a biological signature that is appropriate for removal of the foreign body based on the location and characteristics of the foreign body.

Unlike basal cell carcinoma, malignant melanoma is very rapidly growing skin cancer that frequently can metastasize, leading to fatal outcomes. Malignant melanoma is therefore treated more aggressively. Malignant melanoma is often found in the epidermis 152 (FIG. 3). Malignant melanoma cells may be found in discontiguous patches, unlike basal cell carcinoma which grows in monolithic clusters or nodules in the skin. Multiple passes and/or treatments can be used to amplify the immune response. Each additional pass and/or treatment can be administered after the tissue has already cooled and the immune mediators have been released. In the preferred embodiment, multiple passes are spaced at 0.5 to 10 minute intervals. The interval allows the immune mediators time to reach the target area, but does not allow enough time for them to dissipate significantly. The viable tissue in the regions between injury zones 110 comprise regions where amplification can occur through the further release of immune stimulators. The amplification of the immune response through multiple passes and/or treatments increases the chance of successful cure of aggressive and potentially fatal cancers, such as for example malignant melanoma, at a stage prior to metastasis.

Malignant melanoma treatment by surgical excision doesn't address the problem of potential future recurrence. The present invention provides methods for reducing the likelihood of recurrence of malignant melanomal by treatment with a fractional phototherapy device 100. For example, a vaccine containing melanoma antigens can be injected into the skin or muscle during the same office visit as treatment is performed with a fractional phototherapy device 100. During the initial treatment, the immune system can be focused at the site of melanoma by creating a homing signal by treatment with the fractional phototherapy device 100. This allows for a boosted immune response to the vaccine. Subsequent treatments of the melanoma site with the fractional phototherapy device 100 can be made with or without additional vaccine at 1 to 2 week intervals, or at an interval where the vaccine response curve is near its peak and can be amplified by treatment with the fractional phototherapy device 100 at times near the peak of the immune response. Repeated injury over time can be used to recruit the vaccine-derived immune response to the site of cancer. Thus, a fractional phototherapy device 100 can also serve as a homing device or signal for the host response.

Another potential therapeutic use for the invention is for the treatment of autoimmune disorders that affect the skin and/or visceral organs. For example, alopecia areata represents a condition whereby immune cells of a patient attack and destroy hair follicles. The available immunosuppressive therapies to treat this condition frequently are ineffective, and, in addition, result in significant incidences of systemic side effects. In one embodiment of the invention, the methods and apparatuses disclosed in detail above can be used for the treatment of alopecia areata. The inventive methods are advantageous since a lower incidence of systemic side effects occur. A biological signature effective for the treatment of alopecia areata can be selected. The biological signature can be tailored to counter the characteristic immune response that is responsible for destroying the hair follicle in alopecia areata. Alopecia areata animal models have shown that transfer of CD8+ T-lymphocytes to hair-bearing mice can lead to localized hair loss. On the other hand, transfer of CD4+/CD25+ T-lymphocytes led to a hair loss blockade. Thus, the treatment parameters for the fractional phototherapy device can be selected for example to recruit a predominance of CD4+/CD25+ T-lymphocytes while blocking the recruitment of destructive CD8+ T-lymphocytes.

The process of selection of a particular biological signature is a powerful tool for the treatment of autoimmune diseases. Other examples of autoimmune diseases that can be treated using the methods and apparatuses of the invention include lupus erythematosus, vitiligo, and rheumatoid arthritis. Thus, in one aspect of the invention, the fractional phototherapy treatment around the periphery of a patch of vitiligo can be used to stimulate an immune response that blocks the autoimmune destruction of melanocytes allowing for repigmentation of the treated area. More than one treatment may be required to suspend the autoimmune response against melanocytes.

In another aspect of the invention, immune system overactivity can be locally modulated for the treatment of inflammatory diseases. Inflammatory skin diseases such as for example psoriasis, atopic dermatitis, and acne can be treated by modulating the arm of the immune system which is aberrantly overactive. Inflammatory diseases can also result from immune hyperactivity in visceral organs. For example, inflammatory bowel disease results from immune hyperactivity and can be treated using this invention.

In atopic dermatitis, CD4+ response is overactive, and this leads to the release of ECP and IL-4 and IL-5, which helps increase the concentration of eosinophils. Although eosinophils are useful in the fight against various parasitic infections, these cells often are increased in conditions that involve allergy. Creating a biological signature that selectively increases the CD8+ immune profile can be used to dampen the allergic host response. The methods and apparatuses of the invention can be used for the treatment of atopic dermatitis wherein thermal injury zones capable of achieving such a signature can be created. Similar parameters can be used to treat other inflammatory conditions that require immune suppression of CD4+ overactivity.

In another example of a condition that can be treated by stimulating, resensitizing, or modulating the immune system using a fractional phototherapy device 100 is human papilloma virus (HPV) infection, commonly known as warts. This condition can be difficult to treat medically, especially in acral locations such as the palms and soles, because typical topical therapies alone do not cause a sufficiently vigorous immune response that is specific to the particular HPV strain. By choosing appropriate treatment parameters for the fractional phototherapy device 100, the physician can create a biological signature that is specific to activating cytotoxic T-lymphocytes required for the destruction of viruses, such as for example HPV. Treatment with a fractional phototherapy device 100 can optionally be enhanced by topical application of an agent, such as, imiquimod, cryotherapy, efudex, bleomycin, salicylic acid, and the like.

In another aspect of the invention, methods and apparatuses disclosed herein can be used to modulate the immune response for the treatment of fungal infection. Fungal infections can be treated by choosing treatment parameters to create a biological signature that activates an immune response specific to destruction of fungal organisms. The biological signature can, for example, create biological signatures that activate T-helper cell populations and increase recruitment of eosinophils by eosinophil cationic proteins. Eosinophils can then destroy the fungi that have invaded a tissue. For example, fungal infections of the toenail can be treated using the fractional phototherapy device 100 applied around the nail bed or periungal skin at the periphery of the infected nail.

In yet another aspect of the invention, methods and apparatuses disclosed herein can be used to modulate the immune response for the removal of tattoos, and other biologically inert foreign bodies. Some foreign bodies are not recognized by the immune system and therefore are biologically inert. Examples of biologically inert foreign bodies include tattoo inks and encapsulation materials used for encapsulating tattoo inks. Tattoo inks have been treated with nanosecond pulse lasers that rely on absorption of the laser energy by the ink leading to its fractionation into smaller particles. Not all colors of tattoo inks have sufficient absorption at selected laser wavelengths and this has meant that physicians do not get the desired response or must repeat treatment many times to get adequate results. The patient can thus be subjected to significant pain and a higher risk of scarring. Since the immune system is capable of removing foreign bodies when it is able to detect the presence of the foreign body, the fractional phototherapy device 100 can be used to resensitize the immune system to the tattoo ink and encapsulation material thereby leading to an immune response. The effect of this stimulation is activation of the normal physiological cascade against a foreign body, leading to its effective removal. Thus, noninflammatory skin conditions can be treated using the methods and apparatuses disclosed herein.

In an embodiment, exogenous agents can be used to enhance the inflammatory response that removes the tattoo ink particles. The inflammatory response can be enhanced by using antibodies to suppress growth factors and/or by using cytokines, TNFα, IL-1, and/or IL-6 to stimulate a phagocytic response. A second treatment can be used approximately 7 days following the first treatment to release additional bFGF to promote further collagenlytic activity. Rhanmolipids, agents that inhibit fibroblast and keratinocyte proliferation, can be optionally used to prevent wound contraction and scarring at the end of the treatment.

In another embodiment, stem cells may be administered to the site of treatment, for example, to rejuvenate heart tissue that is either dead or injured. Heart tissue may be treated with fractional phototherapy device 100 to create a particular biological signature that would initiate or accelerate the removal of damaged or dead heart tissue, for example, after a myocardial infarction. Heart stem cells may then be introduced to the treatment site and allow for replacement of the removed tissue.

Although the detailed description contains many specifics, these should not be construed as limiting the scope of the invention but merely as illustrating different examples and aspects of the invention. It should be appreciated that the scope of the invention includes other embodiments not discussed in detail above. Various other modifications, changes and variations which will be apparent to those skilled in the art may be made in the arrangement, operation and details of the method and apparatus of the present invention disclosed herein without departing from the spirit and scope of the invention as defined in the appended claims. Therefore, the scope of the invention should be determined by the appended claims and their legal equivalents. Furthermore, no element, component or method step is intended to be dedicated to the public regardless of whether the element, component or method step is explicitly recited in the claims.

In the claims, reference to an element in the singular is not intended to mean “one and only one” unless explicitly stated, but rather is meant to mean “one or more.” In addition, it is not necessary for a device or method to address every problem that is solvable by different embodiments of the invention in order to be encompassed by the claims.

All printed patents and publications referred to in this application are hereby incorporated herein in their entirety by this reference.

1. A method for modulating the immune response of a subject, the method comprising:
contacting a fractional phototherapy device with a target tissue wherein the immune response is modulated, and wherein the target tissue is skin or a visceral organ.

2. The method of claim 1, wherein the fractional phototherapy device emits energy with a wavelength of about 400 nm to about 28,000 nm.

3. The method of claim 1, wherein the fractional phototherapy device emits energy with a wavelength of about 190 nm to about 400 nm.

4. The method of claim 2, wherein the fractional phototherapy device emits energy with a wavelength of about 1300 nm to about 1900 nm.

5. The method of claim 1, wherein the fractional phototherapy device emits energy with a wavelength of about 2000 nm to about 2400 nm.

6. The method of claim 1, wherein the fractional phototherapy device comprises a Nd:YAG laser.

7. The method of claim 1, wherein the fractional phototherapy device comprises a fiber laser.

8. The method of claim 1, wherein the fractional phototherapy device comprises a CO₂ laser, a holmium laser, or an Er:YAG laser, and combinations thereof.

9. The method of claim 1, wherein the fractional phototherapy device comprises an optical source selected from the group consisting of a flashlamp, a dye laser, a diode laser, and an Er:glass laser, and combinations thereof.

10. The method of claim 1, wherein the immune response is modulated for the treatment of inflammatory disease and the target tissue is a visceral organ.

11. The method of claim 1, wherein the immune response is modulated for the treatment of inflammatory disease and the target tissue is skin.

12. The method of claim 1, wherein the immune response is modulated for the treatment of a noninflammatory disease and the target tissue is skin.

13. The method of claim 1, wherein the immune response is modulated for the treatment of acne or psoriasis.

14. The method of claim 1, wherein the immune response is modulated for the treatment of alopecia areata.

15. The method of claim 1, wherein the immune response is modulated for the treatment of vitiligo.

16. A method for treatment of a subject in need thereof, the method comprising:
contacting a fractional phototherapy device with target tissue having a foreign body, and
modulating the immune response in the target tissue whereby the foreign body is removed, and wherein the target tissue is skin or a visceral organ.

17. The method of claim 16, wherein the target tissue is skin.

18. The method of claim 16, wherein the target tissue is a visceral organ.

19. The method of claim 16, wherein the foreign is completely removed.

20. The method of claim 16, wherein the foreign body is located within dermis of the skin.

21. The method of claim 16, wherein the immune response is amplified by the untreated tissue.

22. The method of claim 16, wherein the immune response is inhibited by the untreated tissue.

23. The method of claim 16, further comprising removal of the foreign body by surgical means.

24. The method of claim 16, further comprising cooling surface of the target tissue.

25. The method of claim 16, further comprising creating a thermal inversion within the target tissue.

26. The method of claim 16, wherein the treatment is proximate to the foreign body.

27. The method of claim 16, wherein the treatment comprising multiple treatments in an interval of about 0.5 minutes to about 10 minutes.

28. The method of claim 16, wherein the treatment creates injury zones.

29. The method of claim 28, wherein the injury zones have a depth to width ratio in the range of about 3:1 to about 10:1.

30. The method of claim 28, wherein the injury zones have a surface area to volume ratio in the range of about 20,000 m⁻¹ to about 100,000 m⁻¹.

31. The method of claim 16, wherein the immune response is localized to within about 10 mm from the target tissue.

32. The method of claim 16, wherein the foreign body comprises skin cancer cells.

33. The method of claim 16, wherein the foreign body comprises tattoo ink particles.

34. The method of claim 16, wherein the foreign body comprises an infection.

35. The method of claim 34, wherein the infection is a fungal infection.

36. The method of claim 16, wherein the foreign body comprises autoimmune disease cells.

37. The method of claim 16, wherein the foreign body comprises human papilloma virus.

38. A method for treatment of a subject in need thereof, the method comprising:
contacting a fractional phototherapy device with target tissue having a foreign body;
administering an exogenous agent to the target tissue; and
modulating the immune response in the target tissue whereby the foreign body is removed, and wherein the target tissue is skin or a visceral organ.

39. The method of claim 38, wherein the exogenous agent is administered topically.

40. The method of claim 38, wherein the exogenous agent is administered systemically.

41. The method of claim 38, wherein the exogenous agent comprises stem cells.

42. The method of claim 38, wherein the exogenous agent comprises homing molecules or targeted stem cells.

43. The method of claim 38, wherein the exogenous agent comprises autologous immunotoxic agents.

44. The method of claim 38, wherein the exogenous agent comprises immunomodulators.

45. The method of claim 38, wherein the exogenous agent comprises a vaccine.

46. The method of claim 38, wherein the exogenous agent comprises cytokines or growth factors, and combinations thereof.

47. The method of claim 38, wherein the exogenous agent comprises paracrine molecules or chemotactic factors, and combinations thereof.
